(19) United States
(12) Patent Application Publication
(10) Pub. No.: US 2010/0076498 A1
(43) Pub. Date: Mar. 25, 2010

(54) Active compression orthopedic screw assembly and method of use

(76) Inventors: Jeff Tyber, Bethlehem, PA (US); Jamy Gannoe, West Milford, NJ (US)

Correspondence Address: WARD & OLIVO, SUITE 300, 382 SPRINGFIELD AVENUE, SUMMIT, NJ, 07901, US

(21) Appl. No.: 12/455,735

(22) Filed: Jun. 5, 2009

Related U.S. Application Data
(60) Provisional application No. 61/131,532, filed on Jun. 10, 2008.

(51) Int. Cl. A61B 17/86

(52) U.S. Cl. 606/302; 606/308; 606/304

(57) ABSTRACT

An orthopedic screw assembly including a proximal member positioned at a proximal end of the orthopedic screw assembly, a distal member positioned at a distal end of the orthopedic screw assembly, and an elongate element having a first end and a second end. The elongate element is coupled to the proximal member at the first end and coupled to the distal member at the second end. The elongate element is configured to apply a dynamic compressive force on each of the proximal member and the distal member, thereby coupling the proximal member to the distal member.

### CROSS-REFERENCE TO RELATED APPLICATION

This application claims the benefit of Provisional Application No. 61/131,532, filed Jun. 10, 2008, the entire contents of which are herein incorporated by reference.

### FIELD OF THE INVENTION

This invention relates to the field of orthopedic implant devices, and more particularly, to an orthopedic screw assembly for providing a dynamic compressive force to secure two or more bone fragments or bones together.

### BACKGROUND OF THE INVENTION

Orthopedic implant devices are often used to repair or reconstruct bones and joints, as well as being used in attaching implants, due to bone fractures, degenerative bone conditions, and similar types of injuries. Frequently, these orthopedic devices require that bone fragments (i.e., the bone is cracked, broken, or cut during a surgical operation such as an osteotomy) must be kept attached together for lengthy periods of time under a sustained force across the fractured site in order to promote healing. As such, these orthopedic implant devices have several functions, for example, to realign bone segments, to apply interfragmental compression to bone fragments or to restore native geometries.

For example, most orthopedic implants are constructed from either one-piece or two-piece members and comprise a threaded distal portion for attaching these implant devices to bone fragments. In addition, these orthopedic implant devices are constructed from standard materials, which undergo normal elastic-plastic mechanical responses during tightening. These orthopedic implants apply initial interfragmental compression, however, due to the biological conditions of bone resorbtion (i.e., removal of bone), which may be sometimes caused from micromotion across lines of fracture, interfragmental compression is lost as implants loosen due to the resorbtion of fragmental contacting surfaces, thereby causing the fragments or device to shorten. This condition eliminates ideal conditions for bone healing, as stated by Wolff's law: bone grows under load and resorbs (i.e. removed) in the lack of loads. Thus, these orthopedic implant devices are not very effective in maintaining interfragmental compression for long periods as is required in order to heal the fracture site.

Other devices utilize elastic wires coupled to a threaded screw to provide compression, yet these too are inefficient, as the coupling means between the elastic wires and the screw is not possible. In addition, they do not allow for accurate placement of guiding wires during installation.

Another device utilizes a coiled spring member to achieve compression of bone fragments. These devices are unable to provide the loads necessary to maintain large compressive loads during application. The springs also have a linear response, which requires large deformations to achieve high loads. Other devices are entirely constructed from an elastic material to achieve compression. These devices may be capable of achieving some compression, but do not have the bending or torsional rigidity to survive over long periods.

In addition, implant loosening is a serious concern and is commonly caused by one or multiple conditions, such as subsidence, centering, fixation loosening or cortical failure to name a few. This has been observed with femoral hip replacements, total ankle and knee replacements, and spinal interbody replacements.

There is therefore a need for an orthopedic implant device assembly and method of use that overcomes some or all of the previously delineated drawbacks of prior orthopedic implant device assemblies.

### SUMMARY OF THE INVENTION

An object of the invention: is to overcome these and other drawbacks of previous inventions.

Another object of the invention is to provide a novel and useful orthopedic assembly that may be utilized to secure multiple bones fragments or bones together.

Another object of the invention is to provide an orthopedic assembly that may be utilized to secure the implant bone interface.

In a first non-limiting aspect of the invention, a orthopedic screw assembly is provided and includes a proximal member positioned at a proximal end of the orthopedic screw assembly; a distal member positioned at a distal end of the orthopedic screw assembly; and an elongate element having a first end and a second end, where the elongate element is coupled to the proximal member at the first end and coupled to the distal member at the second end. The elongate element is configured to apply a dynamic compressive force on each of the proximal member and the distal member, thereby coupling the proximal member to the distal member.

In a second non-limiting aspect of the invention, a method of bone fixation is provided and includes six steps. Step one includes coupling a proximal member to a distal member. Step two comprises providing an elongate member whereby the elongate member comprises a responsive zone of a shape memory material. Step three comprises inserting the elongate member into the proximal member and into the distal member to form a bone fixation assembly. Step four includes inserting a guide wire into bone fragments. Step five includes inserting the guide wire into the bone fixation assembly. Step six includes coupling the fixation assembly to bone fragments so that the responsive zone is positioned adjacent a fracture site in the bone fragments. The responsive zone is adapted to apply a desired compressive force to the bone fragments when coupled thereto.

### BRIEF DESCRIPTION OF THE DRAWINGS

A further understanding of the invention can be obtained by reference to a preferred embodiment set forth in the illustrations of the accompanying drawings. Although the illustrated embodiment is merely exemplary of systems and methods for carrying out the invention, both the organization and method of operation of the invention, in general, together with further objectives and advantages thereof, may be more easily understood by reference to the drawings and the following description. The drawings are not intended to limit the scope of this invention, which is set forth with particularity in the claims as appended or as subsequently amended, but merely to clarify and exemplify the invention.

For a more complete understanding of the invention, reference is now made to the following drawings in which:

FIG. 1 is a perspective view of a screw assembly in accordance with the preferred embodiment of the invention.

FIG. 2 is an unassembled perspective view of the screw assembly, which was shown in FIG. 1.

FIG. 3 is a perspective view of the proximal member, which was shown in FIGS. 1-2 of the preferred embodiment.

FIG. 4 is a perspective view of the distal member, which was shown in FIGS. 1-2 of the preferred embodiment.

FIG. 5 is a perspective view of the elastic member, which was shown in FIG. 2 of the preferred embodiment.

FIG. 6a is a perspective view of the assembled screw assembly being inserted into a bone fracture joint according to the preferred embodiment of the invention.

FIG. 6b is a perspective view of the assembled screw assembly shown in FIG. 6a but with the bone fragments joint coupled under an initial compression.

FIG. 6c is a perspective view of the assembled screw assembly shown in FIGS. 6a and 6b but with the screw assembly applying a dynamic compressive force to the fracture joint.

FIG. 7 is a flow chart, which illustrates the method of coupling the screw assembly shown in FIGS. 1-6c to a bone fracture joint.

FIG. 8 is perspective view of the assembled screw assembly shown in FIGS. 1-5 but with the screw assembly applying a dynamic compressive force to a metal plate.

FIG. 9 is a perspective view of an elastic member in accordance with yet another alternate embodiment of the invention.

FIG. 10 is a perspective view of an elastic member in accordance with another alternate embodiment of the invention.

FIG. 11 is a flow chart, which illustrates the method of manufacturing the elastic member shown in FIGS. 2 and 5.

FIG. 12 is a front view of an elastic member in accordance with an alternate embodiment of the invention.

FIG. 13 is a cross-sectional view of an assembled screw assembly, which utilizes the elastic member shown in FIG. 12.

FIG. 14 is a stress/strain diagram illustrating the properties of the super-elastic member.

### DETAILED DESCRIPTION

The invention may be understood more readily by reference to the following detailed description of preferred embodiment of the invention. However, techniques, systems, and operating structures in accordance with the invention may be embodied in a wide variety of forms and modes, some of which may be quite different from those in the disclosed embodiment. Consequently, the specific structural and functional details disclosed herein are merely representative, yet in that regard, they are deemed to afford the best embodiment for purposes of disclosure and to provide a basis for the claims herein, which define the scope of the invention. It must be noted that, as used in the specification and the appended claims, the singular forms “a”, “an”, and “the” include plural referents unless the context clearly indicates otherwise.

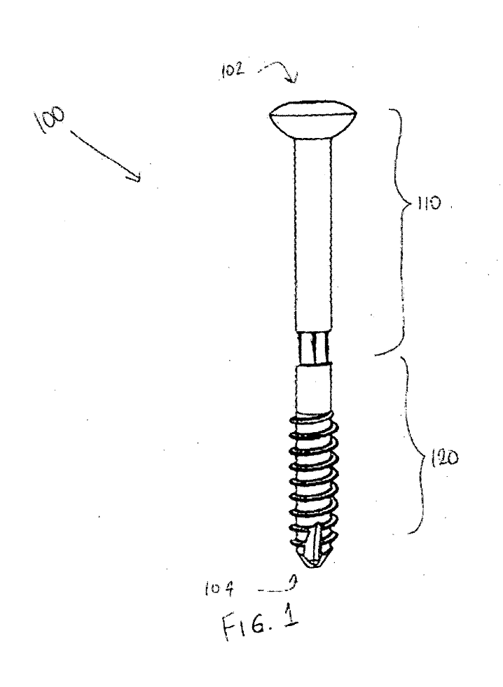

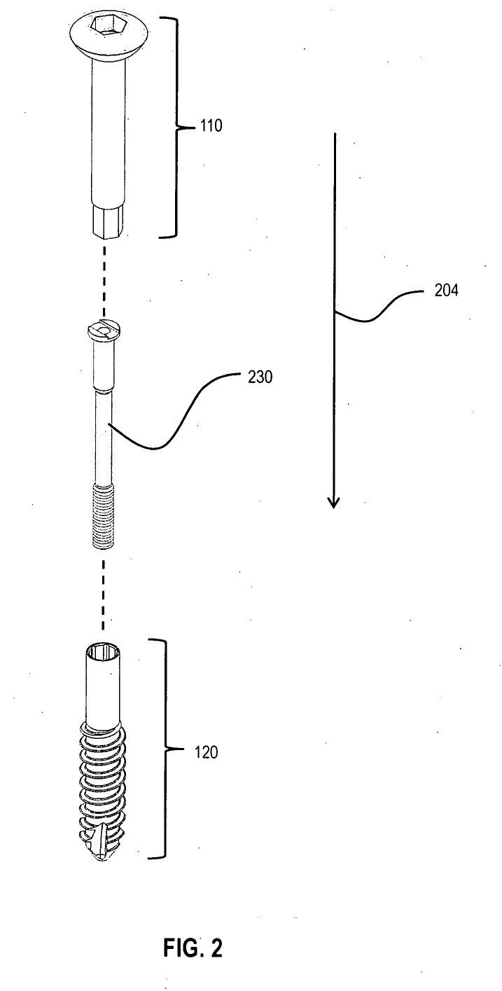

Referring now to FIG. 1, there is shown an orthopedic screw assembly 100 which is made in accordance with the teachings of the preferred embodiment of the invention. As shown, the screw assembly 100 includes a first generally tubular proximal member 110 slideably coupled to a second generally tubular distal member 120 by an elastic member 230 (which will be shown and described in FIG. 2), with the proximal member 110 disposed on a proximal end 102 of the screw assembly 100 and the distal member 120 disposed on the distal end 104 of the screw assembly 100, although in other non-limiting embodiments, proximal member 110 may be coupled to second distal member 120 by a mechanical connection or by substantially any other type of similar connection strategy or technique.

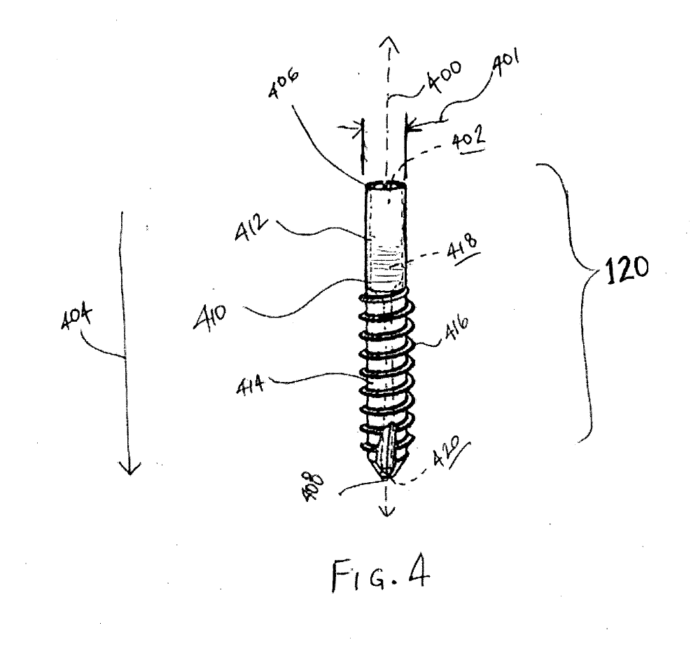

Also, as shown in FIG. 2, proximal member 110 is selectively coupled to a tubular elastic member 230. Yet further, tubular elastic member 230 is selectively coupled to distal member 120, with elastic member 230 being received inside an aperture (which will be shown and described in FIG. 4), with aperture being longitudinally coextensive with distal member 120 in direction 204.

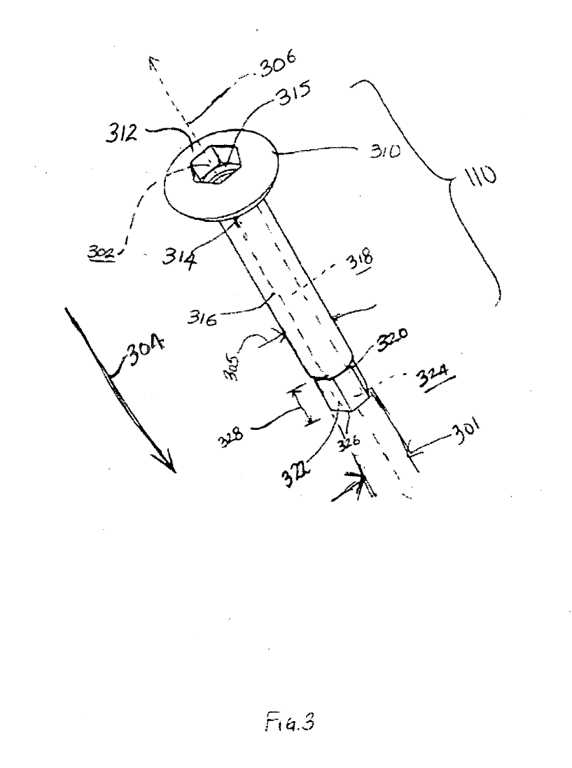

Moreover, and as shown in FIG. 3, proximal member 110 has a first bulbous portion 310 (i.e., portion 310 is generally spherical) with bulbous portion 310 having a generally hexagonal torque transmitting aperture 302 which traverses bulbous portion 310 along longitudinal direction 304 from first end 312 to second end 314, although in other non-limiting embodiments, a star-shaped aperture, a square-shaped aperture, or any other shaped aperture may be utilized. Torque transmitting aperture 302 is utilized to transmit a torque from bulbous portion 310 of proximal member 110 to distal member 120 by receiving a tool having a complementary shape and rotating bulbous portion 310 in an arcuate direction causes proximal member 110 to rotate distal member 120 in the same arcuate direction as proximal member 110 and which will be shown and described later. Also, portion 310 has a second end 314 which terminates into and is rigidly coupled to end 315 of a generally tubular shaft portion 316 (i.e., shaft portion 316 emanates from end 314 in direction 304), with shaft portion 316 enclosing an aperture 318 which is longitudinally coextensive with shaft portion 316 (i.e., inner aperture 318 traverses portion 316 from end 314 to end 320 in direction 304). In another non-limiting embodiments, proximal member 110 has a generally circular washer (not shown). The washer is provided to abut second end 314 with a through-hole in the washer receiving shaft portion 316. The shape of bulbous portion 310 cooperates with the washer (not shown) to prevent proximal member 110 from being pulled through soft-bone when orthopedic screw assembly 110 is coupled to bone. Furthermore, shaft portion 316 emanates and terminates into a generally hexagonal shaped portion 322 (i.e., portion 322 is in the shape of a hexagon having six sides and which is utilized to transmit a torque to distal member 120 which will be described below) having a length 328 and external width 301, with portion 322 fixably coupled to end 320 of portion 322, and with portion 322 enclosing an aperture 324 which is longitudinally coextensive with portion 322 in direction 304. It should be appreciated that portion 322 may be star-shaped, square shaped, or substantially any similar type of shape which may transmit torque by causing portion 322 to engage member 120 and cause distal member 120 to rotate as proximal member 110 is rotated. It should also be appreciated that inner apertures 302, 318 and 324 are aligned along axis 306 so that axis 306 traverses through the center of apertures 302, 318 and 324 so that apertures 302, 318, 324 provide a continuous opening from end 312 to end 326 of portion 322. In operation, apertures 302, 318 and 324 are provided to receive generally elastic member 230 which traverses apertures 302, 318 and 324 and which selectively couples first shaft member 110 to second shaft member 120 and which was shown previously in FIGS. 1-2 and which will be described in detail below.

Further, and as shown in FIG. 4, orthopedic screw assembly 100 comprises a generally tubular distal member 120 with distal member 120 being generally cylindrically shaped from first end 406 to second tapered end 408 (i.e. end 408 has a diameter which is slightly smaller than diameter of end 406). Further, member 120 has a first smooth exterior portion 412 having a generally hexagonally shaped internal aperture 402 (i.e., aperture 402 is in the shape of a hexagon with six edges) which traverses through portion 412 in direction 404 for a length that is at least equal to the length 328 of portion 322, although in other non-limiting embodiments, aperture 402 may be formed of a star shape, a square shape, or any other similar type of shape so as to receive a correspondingly shaped portion 322 in order to transmit torque from proximal member 110 to distal member 120. Also, aperture 402 has an internal diameter 401 which is slightly larger than external diameter 301 of portion 322 of shaft member 110, with portion 322 being received within aperture 402 of portion 412. Also, aperture 402 terminates into an internal aperture 418 having a plurality of circumferentially disposed threads, which have a pitch that is complementary to pitch of threaded portion 510 (which will be shown and described in FIG. 5). Portion 412 terminates into a second generally cylindrical portion 414 which has a plurality of circular threads, such as threads 416, which are circumferentially disposed on the external surface of portion 414. In another non-limiting embodiment, portion 414 has a plurality of machined threads which may be finer pitch threads (not shown) and which are provided to be received in a metal plate (e.g., a metal plate in a prosthesis) in order to couple bone fragments to metal plate. Also, portion 414 has an internal circular aperture 420 which traverses inside portion 414. Internal circular aperture 420 has an internal diameter which is slightly smaller than diameter of aperture 402, and with circular aperture 420 being longitudinally coextensive with portion 414 in direction 404, and with circular aperture 420 aligned with apertures 402 and 418 along axis 400 to form a continuous opening from end 406 to end 408. Also, in one non-limiting embodiment, aperture 402 may traverse through entire longitudinal length of member 120. Aperture 418 is provided to receive threaded portion 510 (which will be shown and described in FIG. 5) of elastic member 230 so that circumferential threads of aperture 418 engage plurality of threads, such as threads 518 (which will be shown and described in FIG. 5) and threadably couple (i.e., mechanically couple) elastic member 230 to distal member 120, which will be shown and described later.

In operation, portion 412 receives portion 322 by positioning portion 322 into aperture 402 of portion 412 and sliding portion 322 downward in direction 404 until edge 320 abuts end 406 and member 110 is restrained from further travel in direction 404, thereby slidably coupling member 110 to member 120.

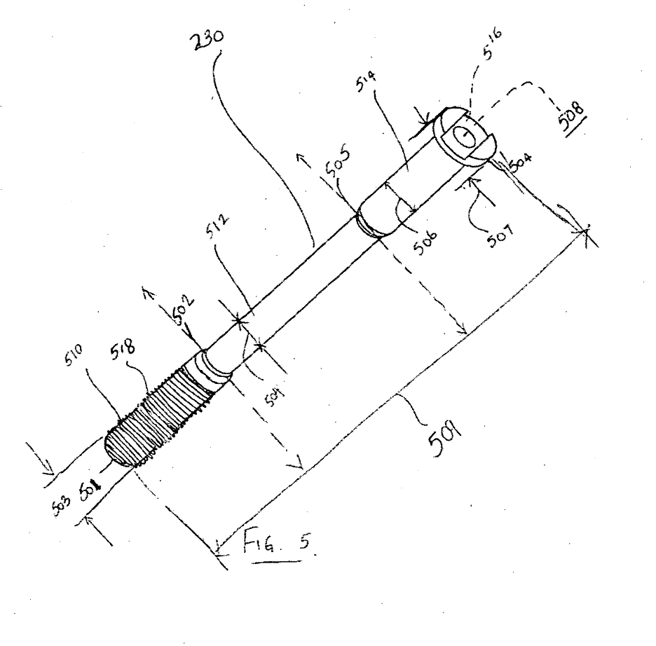

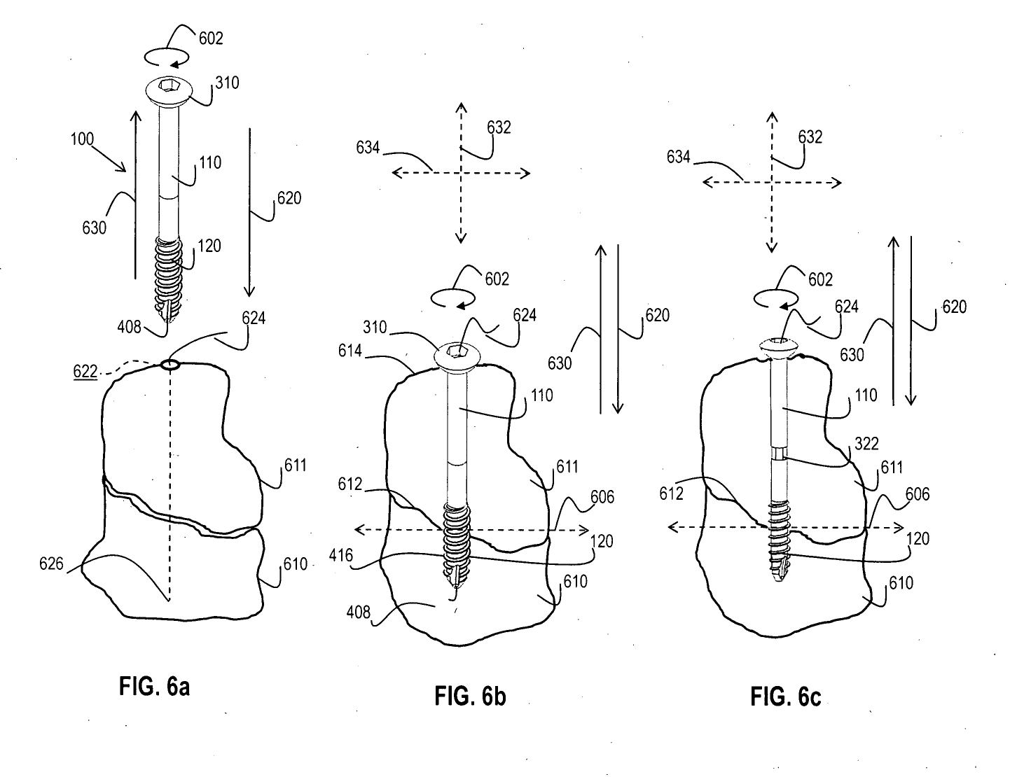

Yet further, and as shown in FIG. 5, orthopedic elastic member 230 is generally tubular from first end 501 to second end 504, and in one non-limiting embodiment, elastic member 230 may be made from a material which undergoes a stress induced martensitic phase transformation (e.g. NiTiNOL or Beta Ti—Nb), although, in other non-limiting embodiments, any elastic member having any elasticity transforming characteristics may be utilized. Member 230 has a first generally cylindrical portion 510, which has a uniform diameter from end 501 to vertical axis 502, and has a plurality of external circumferential threads, such as threads 518, which are provided to threadably couple portion 510 to distal member 120. Also, portion 510 terminates into a second generally cylindrical portion 512, which has a uniform diameter 518 from vertical axis 502 to vertical axis 505, with diameter 518 being slightly smaller than diameter 503 of portion 510. Moreover, portion 512 terminates into a third generally cylindrical portion 514 having a uniform diameter 506 from vertical axis 505 to circular end 504, and with diameter 506 being slightly larger than diameter 518 of portion 512. In one non-limiting embodiment, diameter 506 has the same thickness as diameter 503, although any thickness for diameters 503 and 560 may be utilized for elastic member 230 so long as portion 510 threadably engages threaded aperture 418 of distal member 120. Circular end 504 has a channel 516 which is formed in end 504, with circular end 504 having a diameter 507, which is larger than the diameter 315 of portion 316 of shaft member 110, and diameters 503, 518 and 506 being slightly smaller than the diameter of apertures 318 and 324, thereby causing end 504 to abut end 315 when elastic member 230 is slidably coupled to and is received within apertures 302, 318 and 324 of shaft member 110. Also, elastic member 230 has a through aperture 508 which is longitudinally coextensive with length 509 (i.e., aperture 508 forms a continuous opening from end 504 to end 501), and with aperture 508 being provided to receive a wire (which will be shown and described in. FIG. 6a-6c) to guide orthopedic screw assembly 100 during installation of orthopedic screw assembly 100 into bone fragments or implant attachment. It should be appreciated that the elastic member 230 may be heated at opposed ends 504 and 510 causing the elastic member's 230 stiffness to increase during threading of the threads 518 on the distal end 120 during assembly.

In operation, and as best shown in FIGS. 1, 2 and 6a-6c (which will be described below), the orthopedic screw assembly 100 may be utilized to provide a dynamic compressive force (such as the force caused by stretching elastic member 230) across a fracture site 612 of bone segments 610 and 611, as shown in FIG. 6a. As was previously shown in FIGS. 1 and 2, screw assembly 100 may be assembled by selectively coupling member 110 to member 120 by inserting portion 322 of member 110 into aperture 402 of portion 412 and sliding portion 322 downward in direction 404 until edge 320 abuts end 406 and restrains member 110 from further travel in direction 404. Further, the elastic member 230 is coupled to members 110 and 120 by inserting end 501 into aperture 302 at end 312 of bulbous portion 310 and sliding elastic member 230 in direction 620. Elastic member 230 is selectively inserted into aperture 302 and rotated along arcuate direction 602 which causes member 230 to travel through apertures 316 and 324 of member 110 with plurality of circumferential threads, such as threads 518 engaging complementary internal threads of portion 410, thereby threadably coupling elastic member 230 to distal member 120. Elastic member 230 travels further into aperture 418 of member 120 until end 504 abuts end 315 of member 110 and prevents member 230 from further travel into members 110 and 120, so that elastic member 230 resides within the internal apertures 318 and 324 of member 110 and also within the internal apertures 402 and 418 of member 120, thereby threadably coupling elastic member 230 to member 110 and member 120 and providing an assembled screw assembly 100. It should be appreciated that, in other non-limiting embodiments, screw assembly 100 may be provided to a user in an assembled condition, thereby obviating the need for the user to expend time and energy assembling screw assembly 100.

Further, and as was previously shown in FIGS. 3 and 4, end 322 is complementary to aperture 402 (i.e. each edge on portion 322 has a complementary edge on internal surface of aperture 402 of portion 412), rotating member 110 clockwise or counter-clockwise causes member 120 to also rotate clockwise or counter-clockwise, thereby causing torque to be translated from member 110 to member 120.

Also as shown in FIGS. 6a, the screw assembly 100 may be inserted through a plurality of bone fragments 610 and 611, with bone fragments 610 and 611 being located on opposed ends of bone fracture 612. As shown in FIG. 6a, the screw assembly 100 may be selectively positioned in each of the plurality of bone fragments 610 and 611 by pre-drilling a pilot hole 622 in bone fragments 610 and 611 and inserting a guide wire 624 through predrilled hole 622, and inserting a first end 626 of guide wire 624 to bone segment 610.

Further, and as shown in FIG. 6b, guide wire 624 is inserted into screw assembly 100 by inserting exposed end 626 into end 498 of threaded member 120 and sliding wire 626 through apertures 402, 508, 324 and 302 along direction 630 so that wire 624 protrudes from bulbous portion 310. Screw assembly 100 is inserted into bone segment 611 by positioning end 408 of threaded member 120 in the location of pilot hole 622 and rotating screw assembly 100 along direction of arc 602 to cause screw assembly 100 to travel through bone segment 611 and penetrate into bone segment 610 and across bone fracture 612, while gently pulling on guide wire 624 in direction 630 and feeding guide wire 624 through screw assembly 100. It should be appreciated that plurality of circumferential threads, such as threads 416, are provided so that rotating screw assembly 100 along direction of arc 602 causes the plurality of threads, such as threads 416, to grip or catch the bone segments 610 and 611 and causes the screw assembly 100 to travel into bone segments 610 and 611 in direction 620.

Further, screw assembly 100 is turned along direction of arc 602 until bulbous portion 310 of screw member 110 abuts surface 614 of bone segment 611 and prevents screw member 100 from further travel into bone segment 611. In this state, the screw assembly 100 does not apply any force on elastic member 230, i.e., there is neither any deformation on elastic member 230 along longitudinal axis 632 nor any deformation on member 230 along horizontal axis 634, and the screw assembly is considered to be in an “unloaded” or non-compressive state.

Further, and as shown in FIG. 6c, the screw assembly 100 is further driven (i.e., causing member 120 to penetrate further) into bone segments 610 and 611 by rotating screw assembly 100 along arcuate direction 602 until threaded member 120 further penetrates into bone segments 610 and 611 while portion 310 prevents member 110 from additionally penetrating into bone segment 611, thereby causing the elastic member 230 to be stretched and apply a compressive force on members 110 and 120 while at the same time applying a compressive force on bone segments 610 and 611, and the screw assembly is considered to be in a “stress induced martensite state (i.e., super elastic state). The screw assembly 100 now applies a “constant” force on bone segments 610 and 611 and causes bone segments 610 and 611 to be in a permanently compressive condition, which causes the bone segments 610 and 611 to continue to be drawn toward each other (i.e., bone segment 610 applying a force on bone segment 611 in direction 630 and bone segment 611 applying a force on bone segment 610 in direction 620). It should be appreciated that portion 322, when coupled to member 120, prevents screw assembly 100 from bending along horizontal axis 634 or along longitudinal axis 632 (which is caused by movement of bone segment 610 relative to bone segment 611. It should also be appreciated that portion 322 when coupled to member 120 prevents torsional movement (i.e., movement of member 110 relative to member 120 along direction of arcs 602 and 604) of screw assembly 100 when inserted into bone segments 610 and 611, thereby further increasing the structural integrity of the coupling between bone segments 610 and 611 across bone fracture site 612, as bone segments 610 and 611 may deteriorate along the fracture site 612 over time, or the screw assembly 100 retracts telescopically along the longitudinal axis 632 to continue to apply compressive force.

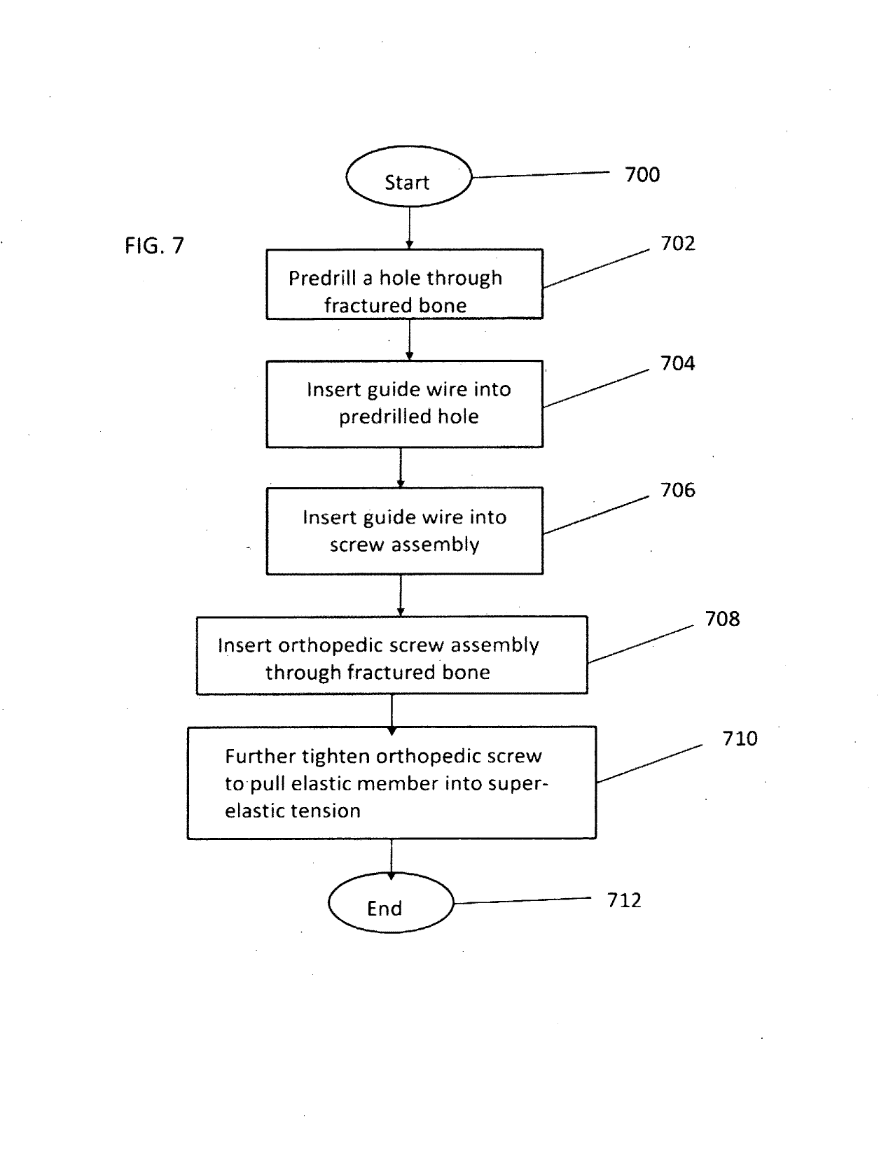

FIG. 7 is a flow chart depicting a method of utilizing the orthopedic screw assembly, such as screw assembly 100, in accordance with the preferred embodiment of the invention. The method starts in step 700 and proceeds to step 702, whereby a hole is predrilled into bone. In step 704, a guide wire is inserted onto the predrilled hole and affixed to the bone segment. In step 706, the guide wire is inserted into the screw assembly 100, and in step 708 the orthopedic screw assembly 100 is inserted into the fractured bone at the location of the predrilled hole. Next, in step 710, the screw assembly 100 is further tightened to pull the elastic member into super-elastic tension and thereby cause a dynamic compression to be exerted on the bone fracture. The method ends in step 712.

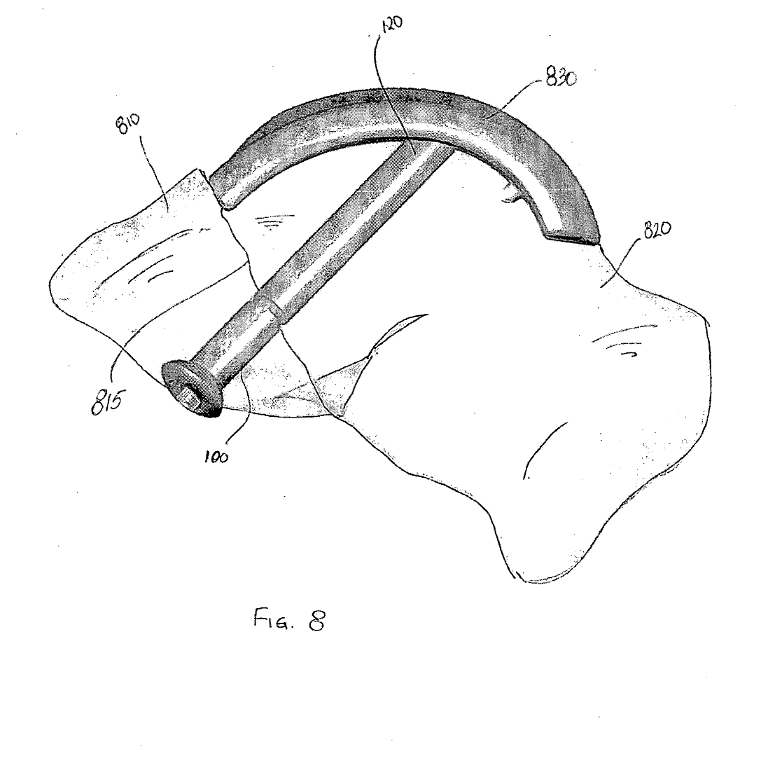

In yet another non-limiting embodiment as shown in FIG. 8, screw assembly 100 may be inserted through a plurality of bone fragments 810 and 820 and through rigid plate member 830 of, in one non-limiting example, a prosthetic joint. Particularly, screw assembly 100 is threadably coupled to bone fragments 810 and 820 across fracture area 815 by inserting screw assembly 100 through bone fragments 810 and 820 and coupling member 120 to plate member 830 by inserting threaded portion 120 of screw assembly 100 into a complementary orifice (not shown) having a corresponding threaded portion to receive threaded portion 120, thereby coupling screw assembly 100 to bone fragments 810 and 820 and to a plate member 830.

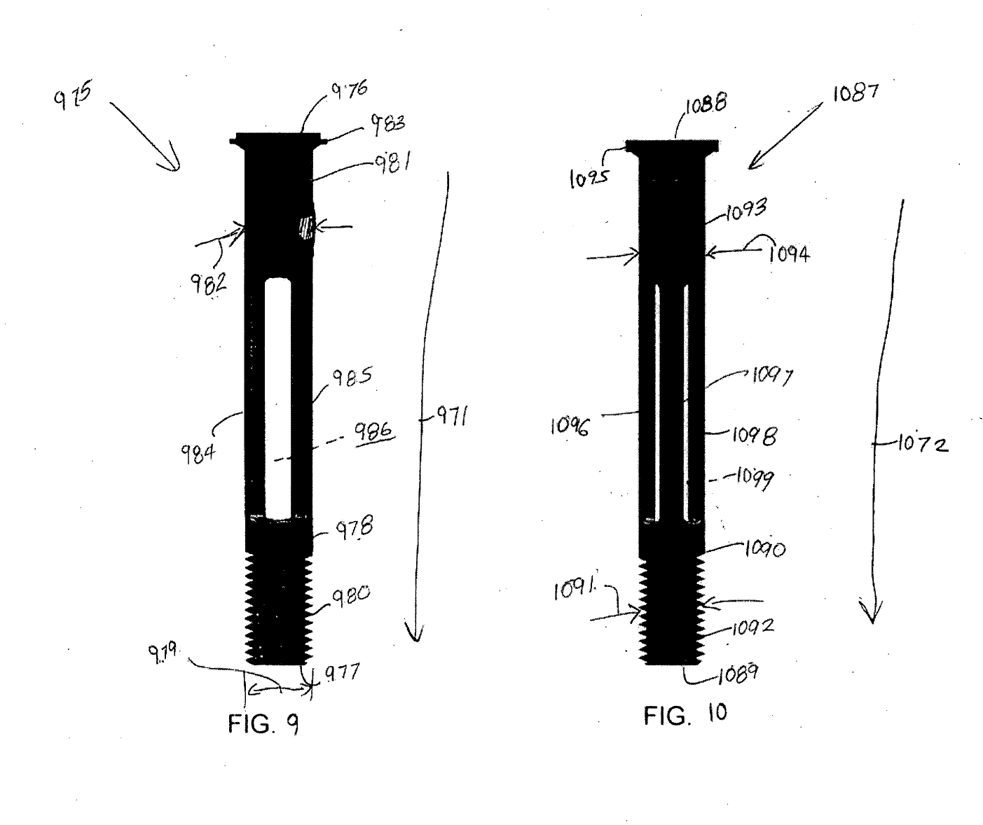

In yet another alternate, although non-limiting embodiment as is shown in FIG. 9, orthopedic screw assembly 100 may comprise a 2-leg elastic member 975 coupled to a distal member 120 while all other features of orthopedic screw assembly 975 remain substantially the same as orthopedic screw assembly 100 as was shown in FIGS. 1-6c.

Particularly, elastic member 975 is generally tubular from first end 976 to second end 977 and encloses a through aperture (not shown) which is longitudinally coextensive from end 976 to end 977 in direction 971, and member 975 has a first generally cylindrical portion 978, which has a uniform diameter 979 and which has a plurality of external circumferential threads, such as threads 980, which are provided to threadably couple elastic member 975 to distal member 120. Also, elastic member 975 has a second generally cylindrical portion 981 having a uniform diameter 982 and which has a circumferential ledge 983 provided at end 976. In one non-limiting embodiment, diameter 982 has the same thickness as diameter 979 of portion 978, although any thickness for diameters 982 and 979 may be utilized for elastic member 975. Further, portions 978 and 981 are coupled to each other by a plurality of substantially similar portions 984 and 985 and which enclose groove 986 which is longitudinally coextensive with portions 984 and 985.

In yet another alternate embodiment as is shown in FIG. 10, orthopedic screw assembly 100 may comprise a 3-leg elastic member 1087 coupled to a distal member 120 while all other features of orthopedic screw assembly 1087 remain substantially the same as orthopedic screw assembly 100 as was shown in FIGS. 1-6c.

Particularly, elastic member 1087 is generally tubular from first end 1088 to second end 1089 and encloses a through aperture (not shown) which is longitudinally coextensive from first end 1088 to second end 1089 in direction 1072, and member 1087 has a first generally cylindrical portion 1090, which has a uniform diameter 1091 and which has a plurality of external circumferential threads, such as threads 1092, which are provided to threadably couple elastic member 1087 to distal member 120. Also, elastic member 1087 has a second generally cylindrical portion 1093 having a uniform diameter 1094 and which has a circumferential ledge 1095 provided at end 1088. In one non-limiting embodiment, diameter 1094 has the same thickness as diameter 1091 of portion 1090, although any thickness for diameters 1094 and 1091 may be utilized for elastic member 1087. Further, portions 1090 and 1093 are coupled to each other by a plurality of substantially similar portions 1096, 1097 and 1098 and which enclose groove 1099 which is longitudinally coextensive with portions 1096, 1097 and 1098.

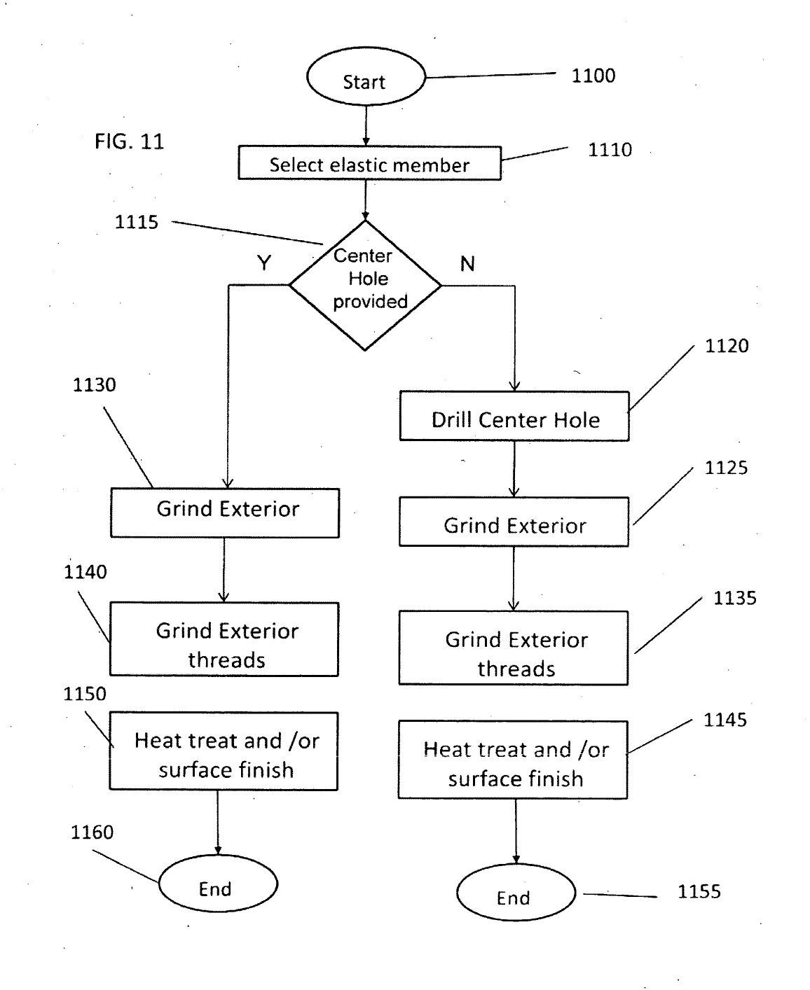

FIG. 11 is a flow chart depicting a method of manufacturing the elastic member, such as elastic member 230, in accordance with an embodiment of the invention. The method starts in step 1100 and proceeds to step 1110, whereby an elastic member, such as elastic member 230 is selected. In step 1115, it is determined if the elastic member 230 has a center hole or aperture which longitudinally traverses the body of the member 230. If an aperture is not provided, a center hole is drilled in step 1120 by gun drilling a center hole, although in other non-limiting examples, a center hole may be machine drilled into elastic member, such as elastic member 230. Next, in step 1125, the exterior surface of the elastic member 230 is ground by electrochemically grinding the surface, although, in other non-limiting examples, the exterior may be ground by utilizing an electro-discharge machine (“EDM” machine). In step 1135, the exterior threaded portion is ground onto the elastic member 230 by electrochemically grinding the elastic member 230, although in other non-limiting examples, an EDM machine may be utilized to provide the exterior threaded portion. Next, in step 1145, the exterior surface is heat treated and/or the exterior surface is. finished. It should be appreciated that the aforementioned steps are non-limiting and, in addition to Electro-Discharge Machining, may also be accomplished by grinding, turning, conventional machining, laser cutting or conventional machining. The method ends in step 1155.

Additionally, if a center hole or aperture is provided in elastic member 230, the method of manufacture proceeds to step 1130, which is substantially the same as step 1125 where the exterior is ground. Next, in step 1140 which is substantially the same as step 1135, the exterior threads are ground into the elastic member 230, and in step 1150 the exterior surface is heat treated and/or the exterior surface is finished, with step 1150 being substantially the same as step 1145. It should be appreciated that the aforementioned steps are non-limiting and, in addition to Electro-Discharge Machining, may also be accomplished by grinding, turning, conventional machining, laser cutting or conventional machining. The method ends in step 1160.

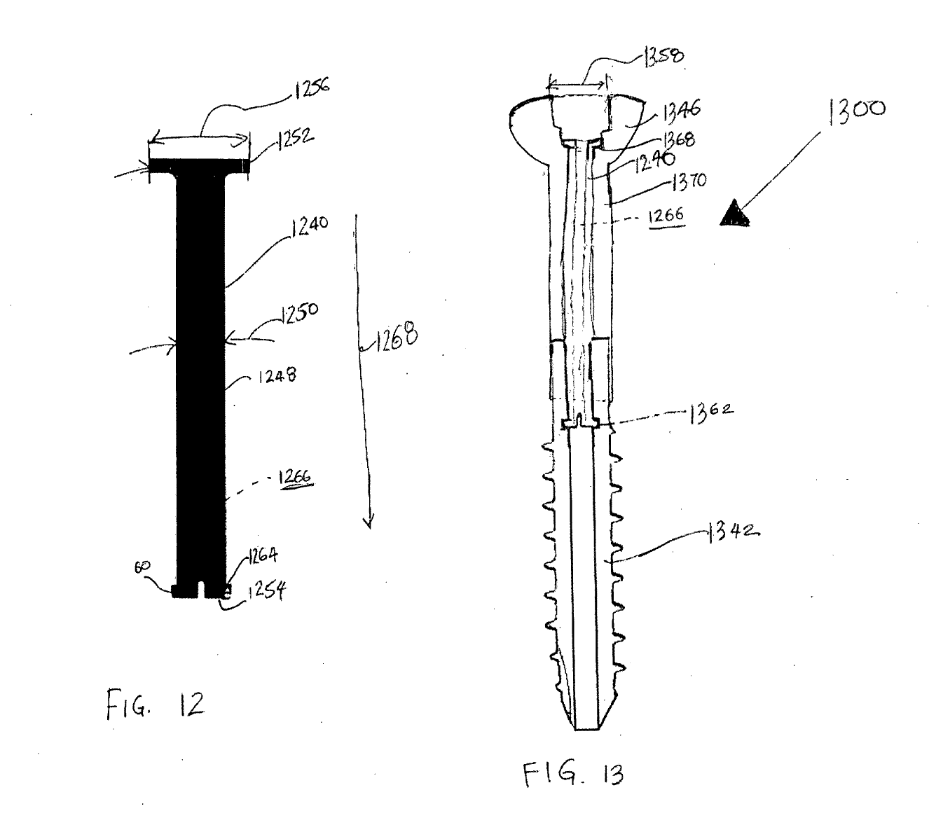

In yet another alternate, although non-limiting embodiment as is shown in FIGS. 12-13, orthopedic screw assembly 1300 may comprise a cantilever elastic member 1240 coupled to a distal member 1342 while all other features of orthopedic screw assembly 1300 remain substantially the same as orthopedic screw assembly 100 as was shown in FIGS. 1-6c.

Particularly, cantilever elastic member 1340 comprises a generally tubular portion 1248 having a uniform thickness 1250 from a first circular end 1252 to a second end 1254. Circular end 1252 has a diameter 1256 which is slightly smaller than diameter 1358 of bulbous portion 1346 and end 1252 is provided to abut edge 1368 of bulbous portion 1346 of proximal member 1370 while second end 1254 is generally circular and has a plurality of cantilever fingers 1260, 1264 which are provided to be received in circumferential groove 1362 of distal member 1342, thereby coupling end 1254 to distal member 1342 and end 1252 to bulbous portion 1346 of proximal member 1370. Additionally, elastic member 1240 comprises a through aperture 1266 which longitudinally traverses elastic member 1240 from first end 1252 to second end 1254 in direction 1268, and which is provided to receive a guide wire for inserting screw assembly 1300 into bone fragments.

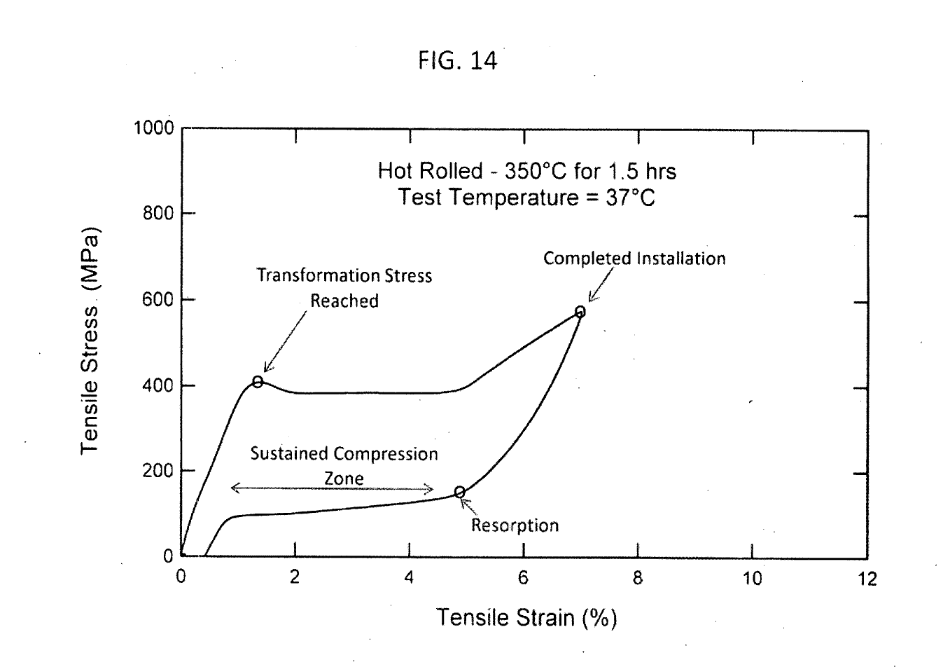

FIG. 14 is a stress/strain diagram illustrating the properties of the elastic member, such as in one non-limiting embodiment, elastic member 230. As shown, an initial increase in deformation strain creates great stresses in the material, followed by a stress plateau with the continued introduction in strain. As the strain is reduced, the stress again plateaus, providing a substantially constant level of stress. This property of the elastic member, such as elastic member 230, allows the member 230 to be subjected to super-elastic compression.

It should be understood that this invention is not limited to the disclosed features and other similar method and system may be utilized without departing from the spirit and the scope of the invention.

While the invention has been described with reference to the preferred embodiment and alternative embodiments, which embodiments have been set forth in considerable detail for the purposes of making a complete disclosure of the invention, such embodiments are merely exemplary and are not intended to be limiting or represent an exhaustive enumeration of all aspects of the invention. The scope of the invention, therefore, shall be defined solely by the following claims. Further, it will be apparent to those of skill in the art that numerous changes may be made in such details without departing from the spirit and the principles of the invention. It should be appreciated that the invention is capable of being embodied in other forms without departing from its essential characteristics.

1. An orthopedic screw assembly, comprising:
a proximal member positioned at a proximal end of the orthopedic screw assembly;
a distal member positioned at a distal end of the orthopedic screw assembly; and
an elongate element having a first end and a second end, wherein the elongate element is coupled to the proximal member at the first end and coupled to the distal member at the second end,
wherein the elongate element is configured to apply a dynamic compressive force on each of the proximal member and the distal member, thereby coupling the proximal member to the distal member.

2. The orthopedic screw assembly of claim 1, wherein the proximal member and the distal member are slideably coupled.

3. The orthopedic screw assembly of claim 2, wherein the proximal member further comprises a partially spherical portion at a third end, a tubular body and a protrusion at a fourth end.

4. The orthopedic screw assembly of claim 3, wherein the protrusion is provided to be coupled to the distal member.

5. The orthopedic screw assembly of claim 4, wherein the protrusion is hexagonal shaped, star shaped, or square shaped.

6. The orthopedic screw assembly of claim 5, wherein the partially spherical portion includes a first orifice, wherein the first orifice is longitudinally coextensive with the partially spherical portion.

7. The orthopedic screw assembly of claim 6, wherein the first orifice has a hexagonal shape, a star shape, or a square shape.

8. The orthopedic screw assembly of claim 7, wherein the first orifice is provided to receive a complementary shaped end of an insertion instrument.

9. The orthopedic screw assembly of claim 8, wherein the proximal member is cannulated having a circular cross-section with the tubular body and with the protrusion.

10. The orthopedic screw assembly of claim 9, wherein the distal member further comprises a first threaded portion positioned on an internal surface at a fifth end, a plurality of bone threads disposed on an outer surface of the distal member, a second orifice at a sixth end, and an aperture longitudinally disposed internally in the distal member.

11. The orthopedic screw assembly of claim 10, wherein the second orifice is coupled to the protrusion on the proximal member, and further wherein the second orifice has a complementary, shape to the protrusion on the proximal member.

12. The orthopedic screw assembly of claim 11, wherein the second orifice has a hexagonal shape, a star shape, or a square shape.

13. The orthopedic screw assembly of claim 12, wherein the plurality of bone threads includes a self-tapping edge, wherein the self-tapping edge provides for removal of bone material during insertion of the orthopedic screw assembly.

14. The orthopedic screw assembly of claim 13, wherein the elongate element further comprises a responsive zone of a shape memory material.

15. The orthopedic screw assembly of claim 14, wherein the elongate element comprises Nitinol.

16. The orthopedic screw assembly of claim 15, wherein the elongate element further comprises a body, a circular portion at the first and a tubular portion at the second end.

17. The orthopedic screw assembly of claim 16, wherein the elongate element is cannulated having a circular cross-section.

18. The orthopedic screw assembly of claim 17, wherein the elongate element is configured to apply a dynamic compressive force on the proximal member and on the distal member, thereby coupling the proximal member to the distal member.

19. The orthopedic screw assembly of claim 18, wherein the tubular portion contains a plurality of machine threads disposed on an outer surface of the tubular portion.

20. The orthopedic screw assembly of claim 19, wherein the plurality of machine threads of the elongate element is threadably coupled to the first threaded portion of the distal member.

21. The orthopedic screw assembly of claim 18, wherein the tubular portion contains a plurality of protrusions positioned at the second end.

22. The orthopedic screw assembly of claim 21, wherein the plurality of protrusions are provided for an interference fit with the distal member, wherein the plurality of protrusions are received in a circumferential groove disposed on an internal surface of the distal member.

23. The orthopedic screw assembly of claim 22, further comprising a washer for coupling to the partially spherical portion of the proximal member, wherein the washer is provided to prevent the partially spherical portion from travelling through soft bone fragments.

24. A method of bone fixation, comprising:
providing a proximal member and a distal member and coupling the proximal member to the distal member;
providing an elongate member comprising a responsive zone of a shape memory material;
inserting the elongate member into the proximal member and into the distal member to form a bone fixation assembly;
inserting a guide wire through bone fragments;
inserting the guide wire through the bone fixation assembly; and
coupling the bone fixation assembly to bone fragments so that the responsive zone is positioned adjacent a fracture site in the bone fragments,
wherein the responsive zone is adapted to apply a desired compressive force to the bone fragments when coupled thereto.

25. The method of claim 24, wherein the proximal member is positioned at a proximal end of the bone fixation assembly.

26. The method of claim 25, wherein the distal member is positioned at a distal end of the bone fixation assembly.

27. The method of claim 26, wherein the proximal member and the distal member are slideably coupled.

28. The method of claim 27, wherein the proximal member further comprises a partially spherical portion at a third end, a tubular body and a protrusion at a fourth end.

29. The method of claim 28, wherein the protrusion is provided to be coupled to the distal member.

30. The method of claim 29, wherein the protrusion is hexagonal shaped, star shaped, or square shaped.

31. The method of claim 30, wherein the partially spherical portion includes a first orifice, wherein the first orifice is longitudinally coextensive with the partially spherical portion.

32. The method of claim 31, wherein the first orifice has a hexagonal shape, a star shape, or a square shape.

33. The method of claim 32, wherein the first orifice is provided to receive a complementary shaped end of an insertion instrument.

34. The method of claim 33, wherein the proximal member is cannulated having a circular cross-section with the tubular body and with the protrusion.

35. The method of claim 34, wherein the distal member further comprises a first threaded portion positioned on an internal surface at a fifth end, a plurality of bone threads disposed on an outer surface of the distal member, a second orifice at a sixth end, and an aperture longitudinally disposed internally in the distal member.

36. The method of claim 35, wherein the second orifice is coupled to the protrusion on the proximal member, and further wherein the second orifice has a complementary shape to the protrusion on the proximal member.

37. The method of claim 36, wherein the second orifice has a hexagonal shape, a star shape, or a square shape.

38. The method of claim 37, wherein the plurality of bone threads includes a self-tapping edge, wherein the self-tapping edge provides for removal of bone material during insertion of the orthopedic screw assembly.

39. The method of claim 38, wherein the elongate element further comprises a responsive zone of a shape memory material.

40. The method of claim 39, wherein the elongate element comprises Nitinol.

41. The method of claim 40, wherein the elongate element further comprises a body, a circular portion at the first and a tubular portion at the second end.

42. The method of claim 41, wherein the elongate element is cannulated having a circular cross-section.

43. The method of claim 42, wherein the elongate element is configured to apply a dynamic compressive force on the proximal member and on the distal member, thereby coupling the proximal member to the distal member.

44. The method of claim 43, wherein the tubular portion contains a plurality of machine threads disposed on an outer surface of the tubular portion.

45. The method of claim 44, wherein the plurality of machine threads of the elongate element is threadably coupled to the first threaded portion of the distal member.

46. The method of claim 43, wherein the tubular portion contains a plurality of protrusions positioned at the second end.

47. The method of claim 46, wherein the plurality of protrusions are provided for an interference fit with the distal member, wherein the plurality of protrusions are received in a circumferential groove disposed on an internal surface of the distal member.

48. The method of claim 47, further comprising coupling a washer to the partially spherical portion of the proximal member, wherein the washer is provided to prevent the partially spherical portion from travelling through soft bone fragments.
